(19) United States
(12) Patent Application Publication
(10) Pub. No.: US 2026/0035320 A1
(43) Pub. Date: Feb. 5, 2026

(54) PROCESS FOR HYDROGENATING A HYDROCARBON STREAM WITH HEAT RECOVERY

(71) Applicant: UOP LLC, Rosemont, IL (US)

(72) Inventors: Ian G. Horn, Streamwood, IL (US); Srinivasan Ramanujam, Bengaluru (IN); Nirlipt Mahapatra, Gurgaon (IN); Gregory R. Werba, Arlington Heights, IL (US)

(21) Appl. No.: 19/248,425

(22) Filed: Jun. 24, 2025

Related U.S. Application Data
(60) Provisional application No. 63/678,043, filed on Jul. 31, 2024.

(51) Int. Cl. C07C 5/10

(52) U.S. Cl. CPC C07C 5/10

(57) ABSTRACT

A process of hydrogenating a hydrocarbon stream is disclosed. The process comprises passing a dehydrogenated feed stream to a hydrogenation reactor. A hydrogen stream is passed to the hydrogenation reactor. In the hydrogenation reactor, the dehydrogenated feed stream is hydrogenated in the presence of hydrogen and a hydrogenation catalyst to produce a hydrogenated effluent stream comprising methylcyclohexane. The hydrogenated effluent stream is cooled by indirect heat exchange with an organic heat exchange fluid stream to provide a vaporized organic heat exchange fluid stream. The vaporized organic heat exchange fluid stream is expanded to provide electrical power. A product stream is separated from a cooled hydrogenated effluent stream.

### FIELD

The field is hydrogenating a hydrocarbon stream. The field may particularly relate to hydrogenating an unsaturated hydrocarbon stream.

### BACKGROUND

Hydrogen is expected to have significant growth potential because it is a clean-burning fuel and can be used in refining processes. However, hydrogen production processes based on steam reforming, autothermal reforming, partial oxidation, or gasification of hydrocarbon or carbonaceous feedstocks are significant emitters of carbon dioxide. Government regulations and societal pressures are increasingly taxing or penalizing carbon dioxide emissions or incentivizing carbon dioxide capture. Consequently, there is significant interest in lowering the cost of hydrogen production and recovering the carbon dioxide byproduct for subsequent sequestration. The recently renewed interest in alternative energy sources and energy carriers opens up new prospects for hydrogen to be applied as a feed for fuel cells, power generation and many more applications.

Increased global demand for hydrogen requires new modes for transporting hydrogen especially to locations that are hydrogen-scarce. Hydrogen generated by renewable energy sources is called green hydrogen. Hydrogen is expected to be an important element in the future fuel economy and may need to be transported to locations as far as 8000 km from the source of generation to remote hydrogen-scarce regions.

There exists a huge regional disparity in the cost for production of hydrogen. A number of technologies have been developed for transporting hydrogen, including ammonia, liquid hydrogen, and liquid organic hydrogen carrier (LOHC) to address this disparity. Toluene-methylcyclohexane (MCH) is expected to be a significant player in LOHC considering its easy integration into the existing fuel sector supply chain and distribution network, utilization of idle refinery assets, flexibility for co-processing, and higher relative safety handling.

LOHC involves the reversible dehydrogenation reaction of a hydrogen carrier such as methylcyclohexane (MCH) to produce toluene and hydrogen. It has been proposed as a solution for the storage, transportation, and distribution of hydrogen produced from renewable or non-renewable energy sources. For power generation, the hydrogen from this process is usually compressed for a downstream power generation unit. Usually, purity requirements for power generation are very stringent. Due to the relatively high cost associated with green hydrogen production, it is necessary to recover almost all of the hydrogen carried for the process to be economical.

MCH can be produced by toluene hydrogenation. One challenge in the toluene hydrogenation process is to efficiently use the excess heat provided by the process.

Accordingly, it would be desirable to have more effective and efficient ways to purify and transport hydrogen while conserving excess heat that is generated.

### BRIEF SUMMARY

A process for hydrogenating a hydrocarbon stream is provided. The process comprises passing a dehydrogenated feed stream to a hydrogenation reactor with a hydrogen stream. In the hydrogenation reactor, the dehydrogenated feed stream is hydrogenated in the presence of hydrogen and a hydrogenation catalyst to produce a hydrogenated effluent stream. The hydrogenated effluent stream is cooled by indirect heat exchange with an organic heat exchange fluid stream to provide a vaporized organic heat exchange fluid stream. The vaporized organic heat exchange fluid stream is expanded to provide electrical power. The electrical power yielded from the expander is greater than yield from a typical process of expanding steam.

### Definitions

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” or “directly” means that fluid flow from the upstream component enters the downstream component without passing through any other intervening vessel.

The term “indirect communication” means that fluid flow from the upstream component enters the downstream component after passing through an intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

The term “Cx” is to be understood to refer to molecules having the number of carbon atoms represented the subscript “x”. Similarly, the term “Cx−” refers to molecules that contain less than or equal to x and preferably x and less carbon atoms. The term “Cx+” refers to molecules with more than or equal to x and preferably x and more carbon atoms.

As used herein, the term “predominant” or “predominate” means greater than 50%, suitably greater than 75% and preferably greater than 90%.

### BRIEF DESCRIPTION OF THE DRAWING

FIG. 1s an illustration of an exemplary embodiment of a process of hydrogenating a hydrocarbons stream.

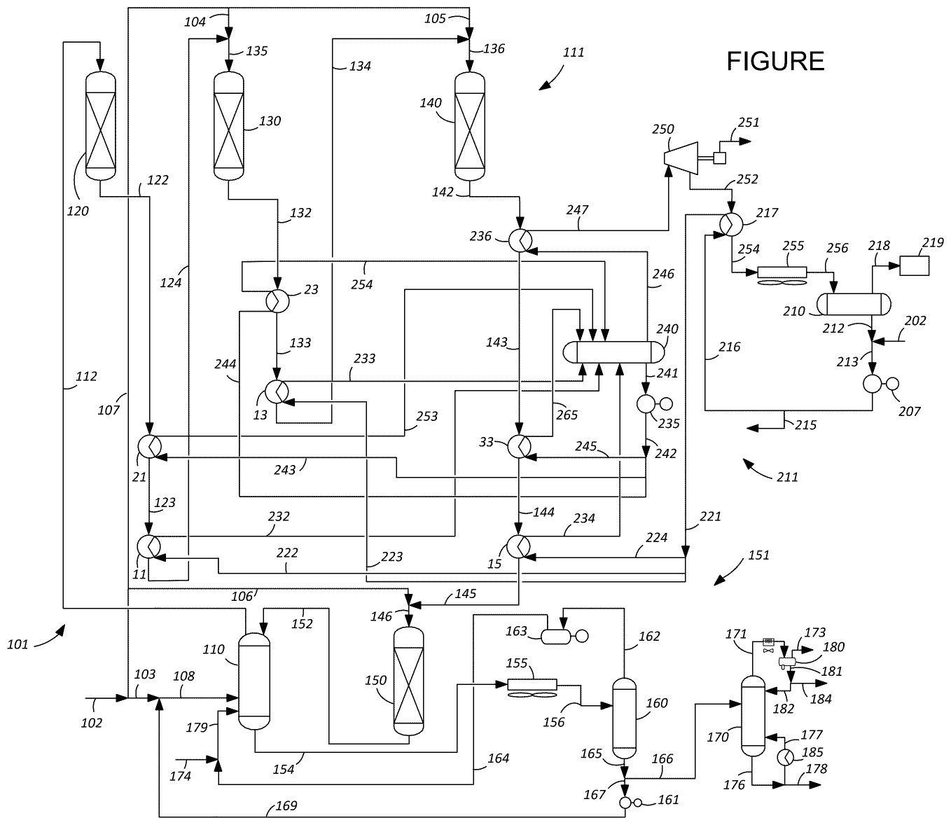

### DETAILED DESCRIPTION

The production and transportation of hydrogen comprises hydrogenation of a dehydrogenated feed stream such as toluene and dehydrogenation of a hydrogenated feed stream such as methylcyclohexane to transport hydrogen. In the hydrogenation process, the dehydrogenated feed stream such as toluene is hydrogenated to a hydrogenated feed stream such as methylcyclohexane with a green hydrogen source. In the dehydrogenation process, the hydrogenated feed stream such as methylcyclohexane is reconverted back to hydrogen and the dehydrogenated feed stream such as toluene at its destination. The hydrogenation process that converts the dehydrogenated feed stream such as toluene to a hydrogenated feed stream is highly exothermic. The present disclosure provides a process of hydrogenating a hydrocarbon stream. The process efficiently utilizes the heat of reaction released in the hydrogenation process. The process comprises an organic Rankin cycle (ORC) to generate electricity from the excess heat of the hydrogenation process rather than the conventional use of steam.

The FIGURE illustrates an embodiment of the process of hydrogenating a hydrocarbon stream comprising an unsaturated hydrocarbon such as toluene. The process 101 comprises a hydrogenation reactor section 111, a heat recovery section 211, and a purification section 151. As shown, a hydrocarbon feed stream comprising an unsaturated hydrocarbon is charged in line 102 to the hydrogenation reactor section 111. The hydrocarbon feed stream in line 102 may be taken from an external source such as a storage tank, a pipeline or a transport vessel (all not shown). If the hydrocarbon feed stream is imported from an external source, such as through a pipeline, land-going vehicle, water-going vehicle, or a storage tank, the feed stream may be exposed to oxygen and require treatment to remove the oxygen and/or oxygenated hydrocarbons, prior to introduction into the hydrogenation reactor section 111.

The oxygen removal treatment may include but is not limited to oxygen stripping, heat soaking, caustic treatment, adsorption using activated alumina and/or molecular sieves, resins, fractionation, clay treatment, or any combination thereof. In an exemplary embodiment, the hydrocarbon feed stream in line 102 may be passed to an oxygen stripping column to remove oxygen, oxygenated hydrocarbons and water from the hydrocarbon feed stream.

In an exemplary embodiment, the hydrogenation reactor section 111 comprises four hydrogenation reactors, a first hydrogenation reactor 120, a second hydrogenation reactor 130, a third hydrogenation reactor 140, and a fourth hydrogenation reactor 150. In another exemplary embodiment, the fourth hydrogenation reactor 150 is a polishing reactor. Fewer or more hydrogenation reactors than four may be utilized.

In an aspect, the hydrocarbon feed stream in line 102 may be fed to a feed header 107 that divides the feed into a first feed stream in line 103, a second feed stream in line 104, a third feed stream in line 105, and a fourth feed stream in line 106.

In an exemplary embodiment, the hydrocarbon feed stream in line 102 is a dehydrogenated feed stream. In an aspect, the dehydrogenated feed stream in line 102 comprises toluene.

The first feed stream is taken in line 103 and passed to a first hydrogenation reactor 120. In an embodiment, the first feed stream in line 103 may be heated by heat exchange with a reactor effluent stream in line 152, as described later in detail, in a combined feed heat exchanger 110. In an aspect, a hydrogen containing stream in line 179 is also heated in the combined feed heat exchanger 110 and passed to the first hydrogenation reactor 120. In an embodiment, the first feed stream in line 103 may be combined with a pumped second hot liquid stream in line 169 to provide a combined first feed stream in line 108 which may be passed to the combined feed heat exchanger 110 to be heated. In an aspect, the hydrogen required for the hydrogenation reaction in the hydrogenation reactor section 111 may be supplied in the hydrogen containing stream in line 179. In another embodiment, the combined first feed stream in line 108 may be combined with the hydrogen containing stream in line 179 and passed to the combined feed heat exchanger 110 to be heated.

A heated first combined feed stream is discharged in line 112 from the combined feed heat exchanger 110 and charged to the first hydrogenation reactor 120. In the first hydrogenation reactor 120, the dehydrogenated hydrocarbon such as toluene present in the first feed stream is hydrogenated in the presence of hydrogen and a hydrogenation catalyst to produce a first hydrogenated effluent stream comprising a hydrogenated hydrocarbon such as methylcyclohexane. The first hydrogenated effluent stream comprising the hydrogenated hydrocarbon such as methylcyclohexane is discharged in line 122 from the first hydrogenation reactor 120.

Any suitable hydrogenation catalysts may be used in the first hydrogenation reactor 120. The hydrogenation catalyst should have high selectivity and a low rate of coke lay down. Suitable hydrogenation catalysts for the first hydrogenation reactor 120 may include, but are not limited to, a metal of Group VIII of the Periodic Table and optionally a metal of Group I of the Periodic Table. Suitable hydrogenation catalysts for the first hydrogenation reactor 120 may also include, but are not limited to, 0.05 wt % to 30 wt % of a metal of Group VIII of the Periodic Table and optionally 0.1 wt % to 3 wt % of a metal of Group I of the Periodic Table.

The first hydrogenation reactor 120 may be operated at a pressure of about 1034 kPa(g) (150 psig) to about 6895 kPa(g) (1000 psig), or about 2068 kPa(g) (300 psig) to about 3447 kPa(g) (500 psig). The first hydrogenation reactor 120 may be operated at an inlet temperature of about 204° C. (400° F.) to about 232° C. (450° F.). The first hydrogenation reactor 120 may be operated at an outlet temperature of about 232° C. (450° F.) to about 371° C. (700° F.).

The hydrogenation of the dehydrogenated hydrocarbon such as toluene in the first hydrogenation reactor 120 in the presence of the hydrogenation catalyst is performed at a relatively high reaction temperature. The first hydrogenated effluent stream in line 122 exits the first hydrogenation reactor 120 at a high temperature. Heat can be recovered from the first hydrogenated effluent stream in line 122 which may be utilized in the process 101 or exported to other locations.

In an exemplary embodiment, the heat recovery section 211 comprises an organic heat exchange fluid, a heat exchange fluid separator 240, an expander 250 which may be a turbine-generator, an economizer 217, a cooler 255, and a receiver 210.

An organic heat exchange fluid stream in line 234, as described later in detail, is supplied to the heat exchange fluid separator 240. In an embodiment, the organic heat exchange fluid stream in line 234 may comprise a dehydrogenated hydrocarbon. In an exemplary embodiment, the organic heat exchange fluid stream in line 234 and the hydrocarbon feed stream in line 102 may both comprise the same dehydrogenated hydrocarbon. A liquid organic heat exchange fluid stream is taken from the heat exchange fluid separator 240 in line 241. The liquid organic heat exchange fluid stream in line 241 is heat exchanged with one or more hydrogenated effluent streams from the hydrogenation reactor section 111 to recover heat. The liquid organic heat exchange fluid stream in line 241 may be passed through a pump 235 to provide a pumped liquid organic heat exchange fluid stream in line 242 which is passed to the hydrogenation reactor section 111.

In an embodiment, the pumped liquid organic heat exchange fluid stream in line 242 is divided into a first liquid organic heat exchange fluid stream in line 243, a second liquid organic heat exchange fluid stream in line 244, and a third liquid organic heat exchange fluid stream in line 245.

The first hydrogenated effluent stream in line 122 is cooled by heat exchange with the first liquid organic heat exchange fluid stream in line 243 in a first vaporizer 21. A vaporized first organic heat exchange fluid stream is discharged in line 253 from the first vaporizer 21. The first hydrogenated effluent stream in line 122 is at an elevated temperature of about the outlet temperature of the first hydrogenation reactor 120. In the first vaporizer 21, the first hydrogenated effluent stream in line 122 is indirectly heat exchanged with the first liquid organic heat exchange fluid stream in line 243 and the heat of the first hydrogenated effluent stream vaporizes the first liquid organic heat exchange fluid stream. The vaporized first organic heat exchange fluid stream in line 253 is recycled back to the heat exchange fluid separator 240.

A cooled first hydrogenated effluent stream is discharged in line 123 from the first vaporizer 21. The cooled first hydrogenated effluent stream in line 123 is at an elevated temperature and further heat can be recovered from it. The cooled first hydrogenated effluent stream in line 123 is passed to a first effluent cooler 11 to release further heat which is recovered. In an embodiment, a first organic heat exchange fluid stream in line 222 is taken from the organic heat exchange fluid stream in line 221 and passed to the first effluent cooler 11 to indirectly heat exchange with the cooled first hydrogenated effluent stream in line 123 and absorb the heat from the cooled first hydrogenated effluent stream. A heated first organic heat exchange fluid stream is discharged from the first effluent cooler 11 in line 232. The heated first organic heat exchange fluid stream in line 232 is sent to the heat exchange fluid separator 240.

A twice cooled first hydrogenated effluent stream is discharged in line 124 from the first effluent cooler 11. The twice cooled first hydrogenated effluent stream in line 124 is charged to the second hydrogenation reactor 130. In an embodiment, the twice cooled first hydrogenated effluent stream in line 124 is combined with the second feed stream in line 104 to provide a combined second feed stream in line 135 which is charged to the second hydrogenation reactor 130. In the second hydrogenation reactor 130, dehydrogenated hydrocarbon such as toluene present in the second feed stream in line 104 and in the twice cooled first hydrogenated effluent stream in line 124 is hydrogenated in the presence of hydrogen and a hydrogenation catalyst to produce a second hydrogenated effluent stream comprising a hydrogenated hydrocarbon such as methylcyclohexane. Hydrogen for the second hydrogenation reaction in the second hydrogenation reactor 130 may be present in the first hydrogenated effluent stream in line 122. Fresh make-up hydrogen may be supplemented to the second hydrogenation reactor 130, but this is not preferred. The second hydrogenated effluent stream comprising the hydrogenated hydrocarbon such as methylcyclohexane is discharged in line 132 from the second hydrogenation reactor 130.

Any suitable hydrogenation catalysts may be used in the second hydrogenation reactor 130. The second hydrogenation reactor 130 may comprise one or more of the hydrogenation catalyst as previously described. The second hydrogenation reactor 130 may comprise a similar or a different hydrogenation catalyst than the first hydrogenation reactor 120.

The second hydrogenation reactor 130 may be operated at a pressure of about 1034 kPa(g) (150 psig) to about 6895 kPa(g) (1000 psig), or about 2068 kPa(g) (300 psig) to about 3447 (500 psig). The second hydrogenation reactor 130 may be operated at an inlet temperature of about 204° C. (400° F.) to about 232° C. (450° F.). The second hydrogenation reactor 130 may be operated at an outlet temperature of about 232° C. (450° F.) to about 371° C. (700° F.). The second hydrogenation reactor 130 may be operated at similar or different operating pressure and temperature than the first hydrogenation reactor 120.

The second hydrogenated effluent stream in line 132 exits the second hydrogenation reactor 130 at an elevated temperature of about the outlet temperature of the second hydrogenation reactor 130. In an aspect, the second hydrogenated effluent stream in line 132 exits the second hydrogenation reactor 130 at the same temperature at which the first hydrogenated effluent stream in line 122 exits the first hydrogenation reactor 120. In another aspect, the second hydrogenated effluent stream in line 132 may exit the second hydrogenation reactor 130 at a lower temperature than the temperature at which the first hydrogenated effluent stream in line 122 exits the first hydrogenation reactor 120. In yet another aspect, the second hydrogenated effluent stream in line 132 may exit the second hydrogenation reactor 130 at a higher temperature than the temperature at which the first hydrogenated effluent stream in line 122 exits the first hydrogenation reactor 120. Heat can be recovered from the second hydrogenated effluent stream in line 132 which may be utilized in the process 101 or exported to other locations.

The second hydrogenated effluent stream in line 132 is cooled by indirect heat exchange with the second liquid organic heat exchange fluid stream in line 244 in a second vaporizer 23. A vaporized second organic heat exchange fluid stream is taken in line 254 from the second vaporizer 23. The second hydrogenated effluent stream in line 132 is at an elevated temperature of about the outlet temperature of the second hydrogenation reactor 130. In the second vaporizer 23, the second hydrogenated effluent stream in line 132 is indirectly heat exchanged with the second liquid organic heat exchange fluid stream in line 244 and the heat of the second hydrogenated effluent stream vaporizes the second liquid organic heat exchange fluid stream. The vaporized second organic heat exchange fluid stream in line 254 is recycled back to the heat exchange fluid separator 240.

A cooled second hydrogenated effluent stream is discharged from the second vaporizer 23 in line 133. The cooled second hydrogenated effluent stream in line 133 is still at an elevated temperature and further heat can be recovered from it. The cooled second hydrogenated effluent stream in line 133 is passed to a second effluent cooler 13 to release further heat which is recovered. In an embodiment, a second organic heat exchange fluid stream in line 223 is taken from the organic heat exchange fluid stream in line 221 and passed to the second effluent cooler 13 to indirectly heat exchange with the cooled second hydrogenated effluent stream in line 133 and absorb the heat from the cooled second hydrogenated effluent stream. A heated second organic heat exchange fluid stream is discharged from the second effluent cooler 13 in line 233. The heated second organic heat exchange fluid stream in line 233 is sent to the heat exchange fluid separator 240.

A twice cooled, second hydrogenated effluent stream is discharged in line 134 from the second effluent cooler 13. The twice cooled, second hydrogenated effluent stream in line 134 is charged to the third hydrogenation reactor 140. In an embodiment, the twice cooled, second hydrogenated effluent stream in line 134 is combined with the third feed stream in line 105 to provide a combined third feed stream in line 136 which is charged to the third hydrogenation reactor 140. In the third hydrogenation reactor 140, a dehydrogenated hydrocarbon such as toluene present in the third feed stream in line 105 and in the twice cooled second hydrogenated effluent stream in line 134 is hydrogenated in the presence of hydrogen and a hydrogenation catalyst to produce a third hydrogenated effluent stream comprising a hydrogenated hydrocarbon such as methylcyclohexane. The third hydrogenated effluent stream comprising the hydrogenated hydrocarbon such as methylcyclohexane is discharged in line 142 from the third hydrogenation reactor 140. Hydrogen for the third hydrogenation reaction in the third hydrogenation reactor 140 may be present in the second hydrogenated effluent stream in line 132. Fresh make-up hydrogen may be supplemented to the third hydrogenation reactor 130, but this is not preferred. Any suitable hydrogenation catalysts may be used in the third hydrogenation reactor 140. The third hydrogenation reactor 140 may comprise one or more of the hydrogenation catalyst as previously described. The third hydrogenation reactor 140 may comprise a similar or a different hydrogenation catalyst than the first hydrogenation reactor 120 and/or the second hydrogenation reactor 130.

The third hydrogenation reactor 140 may be operated at a pressure of about 1034 kPa(g) (150 psig) to about 6895 kPa(g) (1000 psig), or about 2068 kPa(g) (300 psig) to about 3447 (500 psig). The third hydrogenation reactor 140 may be operated at an inlet temperature of about 204° C. (400° F.) to about 232° C. (450° F.). The third hydrogenation reactor 140 may be operated at an outlet temperature of about 232° C. (450° F.) to about 371° C. (700° F.). The third hydrogenation reactor 140 may be operated at similar or different operating pressure and temperature than the first hydrogenation reactor 120 and/or the second hydrogenation reactor 130.

The third hydrogenated effluent stream in line 142 exits the third hydrogenation reactor 140 at an elevated temperature of about the outlet temperature of the third hydrogenation reactor 140. In an aspect, the third hydrogenated effluent stream in line 142 exits the third hydrogenation reactor 140 at the same temperature at which the second hydrogenated effluent stream in line 132 exits the second hydrogenation reactor 130. In another aspect, the third hydrogenated effluent stream in line 142 may exit the third hydrogenation reactor 140 at a lower temperature than the temperature at which the second hydrogenated effluent stream in line 132 exits the second hydrogenation reactor 130. In yet another aspect, the third hydrogenated effluent stream in line 142 may exit the third hydrogenation reactor 140 at a higher temperature than the temperature at which the second hydrogenated effluent stream in line 132 exits the second hydrogenation reactor 130. Heat can be recovered from the third hydrogenated effluent stream in line 142 which may be utilized in the process 101 or exported to other locations.

The third hydrogenated effluent stream in line 142 may be cooled by heat exchange with the third liquid organic heat exchange fluid stream in line 245 in a third vaporizer 33. In an embodiment, before passing to the vaporizer and/or the cooler, the third hydrogenated effluent stream in line 142 may be first cooled by heat exchange in a superheater 236 with a vapor organic heat exchange fluid stream discharged in line 246 from the heat exchange fluid separator 240. Any of the first hydrogenated effluent stream in line 122, the second hydrogenated effluent stream in line 132 and the third hydrogenated effluent stream in line 142 may be first cooled in a superheater 236 before passing it to the respective vaporizer and/or the cooler to provide a superheated vapor stream. In a preferred embodiment, the superheater 236 is provided at the location at which the temperature and the mass flow rate of the hydrogenated effluent stream from a hydrogenation reactor section 111 is highest. The third hydrogenated effluent stream in line 142 may be directly passed to the superheater 236.

In an exemplary embodiment, the superheater 236 is provided downstream of the third hydrogenation reactor to first cool the third hydrogenated effluent stream in line 142 with the vapor organic heat exchange fluid stream discharged in line 246 from the heat exchange fluid separator 240 to provide a superheated vaporized organic heat exchange fluid stream in line 247. In the embodiment illustrated in the FIGURE, the superheater 236 is in direct downstream fluid communication with the third hydrogenation reactor 140. A cooled third hydrogenated effluent stream is discharged in line 143 from the superheater 236.

The cooled third hydrogenated effluent stream in line 143 has surplus heat which may be further recovered. The cooled third hydrogenated effluent stream in line 143 is further cooled by heat exchange with a third liquid organic heat exchange fluid stream in line 245 in a third vaporizer 33. In the third vaporizer 33, the cooled third hydrogenated effluent stream in line 143 is indirectly heat exchanged with the third liquid organic heat exchange fluid stream in line 245 and the heat of the cooled third hydrogenated effluent stream vaporizes the third liquid organic heat exchange fluid stream. The vaporized third organic heat exchange fluid stream is taken in line 265 and recycled back to the heat exchange fluid separator 240.

A twice cooled third hydrogenated effluent stream is discharged in line 144 from the third vaporizer 33. The twice cooled third hydrogenated effluent stream in line 144 is still at an elevated temperature and further heat can be recovered from it. The twice cooled third hydrogenated effluent stream in line 144 is passed to a third effluent cooler 15 to release further heat which is recovered. In an embodiment, a third organic heat exchange fluid stream in line 224 is taken from the organic heat exchange fluid stream in line 221 and passed to the third effluent cooler 15 to heat exchange with the twice cooled third hydrogenated effluent stream in line 144 and absorb the heat from the twice cooled third hydrogenated effluent stream. A heated third organic heat exchange fluid stream is discharged from the third effluent cooler 15 in line 234. The heated third organic heat exchange fluid stream in line 234 is sent to the heat exchange fluid separator 240.

The heated first organic heat exchange fluid stream in line 232, the heated second organic heat exchange fluid stream in line 233, the heated third organic heat exchange fluid stream in line 234, the vaporized first organic heat exchange fluid stream in line 253, the vaporized second organic heat exchange fluid stream in line 254, and the vaporized third organic heat exchange fluid stream in line 265, all are recycled back to the heat exchange fluid separator 240. In the heat exchange fluid separator 240, all heated organic heat exchange fluid streams and all vaporized organic heat exchange fluid streams are separated to provide the vapor organic heat exchange fluid stream in line 246 and the liquid organic heat exchange fluid stream in line 241. The heated first organic heat exchange fluid stream in line 232, the heated second organic heat exchange fluid stream in line 233, the heated third organic heat exchange fluid stream in line 234 are typically liquid streams.

Although the FIGURE shows one heat exchange fluid separator 240 for separating all the heated organic heat exchange fluid streams and all vaporized organic heat exchange fluid streams, there may be a dedicated heat exchange fluid separator 240 for each of the heated organic heat exchange fluid streams and their corresponding vaporized organic heat exchange fluid streams. So, there may be one dedicated heat exchange fluid separator 240 for the heated first organic heat exchange fluid stream in line 232 and the vaporized first organic heat exchange fluid stream in line 253, one dedicated heat exchange fluid separator 240 for the heated second organic heat exchange fluid stream in line 233 and the vaporized second organic heat exchange fluid stream in line 254, and one dedicated heat exchange fluid separator 240 for the heated third organic heat exchange fluid stream in line 234 and the vaporized third organic heat exchange fluid stream in line 265.

The organic heat exchange fluid stream absorbs the surplus heat of the hydrogenated effluent stream. The surplus heat boils the organic heat exchange fluid stream into a vaporized organic heat exchange fluid stream. This heat is converted to electrical power in the heat recovery unit 211.

Referring to the superheater 236, the superheated vaporized organic heat exchange fluid stream is discharged in line 247 from the superheater 236. Heat is recovered from the superheated vaporized organic heat exchange fluid stream in line 247 in the heat recovery unit 211 and converted to power. In accordance with the present disclosure, the superheated vaporized organic heat exchange fluid stream in line 247 is passed to the expander 250. In an exemplary embodiment, the expander 250 is a turbine-generator. In the expander 250, the superheated vaporized organic heat exchange fluid stream in line 247 undergoes an isentropic expansion which may be harnessed by turning the turbine to generate electrical power 251. An expanded organic heat exchange fluid stream comprising a vapor-liquid mixture is discharged in line 252 from the expander 250. The expanded organic heat exchange fluid stream in line 252 is cooled in an economizer 217 by heat exchange with an organic heat exchange fluid stream in line 216. A cooled organic heat exchange fluid stream in line 254 is passed through a cooler such as an air cooler 255 to condense it. A condensed organic heat exchange fluid stream is discharged in line 256 from the cooler 255 and passed to the receiver 210. In an embodiment, the receiver 210 may be operated at vacuum pressure.

As the receiver 210 operates at a vacuum, it is possible that there may be air present in the system. In an aspect, a vacuum pump 219 may be provided on line 218 from the receiver 210 to remove any such air, nitrogen, or non-condensable that may get into the system. A vacuum may be pulled on line 218 with the vacuum pump 219 to generate vacuum in the receiver 210. A liquid portion is discharged in line 212 from the receiver 210. The liquid portion in line 212 is recycled back into the heat recovery unit 211 to absorb heat from the one or more hydrogenated effluent streams of the hydrogenation reactor section 111.

In accordance with the present disclosure, the heat of reaction is recovered from the hydrogenated effluent stream from the hydrogenation reactor section 111 to power an Organic Rankine Cycle (ORC) machine, which uses an organic fluid such as halohydrocarbons or hydrocarbons or both instead of water or other fluid to generate power. In an embodiment, the heat of reaction is recovered from hydrogenated effluent stream from the hydrogenation reactor section 111 in the heat recovery section 211 operating as an ORC machine.

In an embodiment, an organic heat exchange fluid feed stream is taken in line 202 and passed to the heat recovery unit 211. In an aspect, the organic heat exchange fluid feed stream in line 202 is combined with the liquid portion in line 212 to provide a combined organic heat exchange fluid feed stream in line 213. The combined organic heat exchange fluid feed stream in line 213 may be pumped to the hydrogenation reactor section 111 through a pump 207. To prevent impurities from building up in the loop, a purge stream may be taken in line 215 from the combined organic heat exchange fluid feed stream in line 213. After purge, the combined organic heat exchange fluid feed stream may be transported in line 216 and heated by heat exchange with the expanded organic heat exchange fluid stream in line 252 in the economizer 217. A heated organic heat exchange fluid feed stream is discharged in line 221 from the economizer 217. The heated organic heat exchange fluid feed stream in line 221 is passed to the hydrogenation reactor section 111 to recover surplus heat from the hydrogenated effluent stream. In accordance with the present disclosure, the heated organic heat exchange fluid feed stream in line 221 is divided into the first organic heat exchange fluid stream in line 222, the second organic heat exchange fluid stream in line 223, and the third organic heat exchange fluid stream in line 224 which are passed to the hydrogenation reactor section 111 to recover surplus heat from the hydrogenated effluent streams as previously described.

In an embodiment, the organic heat exchange fluid in the feed stream in line 202 may comprise hydrogenated effluent from the hydrogenation reactors such as methylcyclohexane or even the dehydrogenated feed to the reactors such as toluene or a mixture thereof. In an exemplary embodiment, the organic heat exchange fluid in the feed stream in line 202 is toluene. In a typical heat recovery unit, steam is used to recover heat from the reactor effluent and convert it into the electrical power in the turbine generator. Applicants found that using toluene as the organic heat exchange fluid stream in line 202 produces more power than steam. Typically, the heat of vaporization of water is about 2258 kJ/kg, and the heat of vaporization of toluene is about 365 kJ/kg. Toluene may need a higher mass flow rate of about four times the steam flow rate due to the low heat of vaporization as compared to the water. However, toluene requires less volume to expand than steam in the expander 250.

In another exemplary embodiment, the organic heat exchange fluid in the feed stream in line 202 is methylcyclohexane. In yet another exemplary embodiment, the organic heat exchange fluid in the feed stream in line 202 is a mixture of toluene and methylcyclohexane.

In accordance with the present disclosure, the organic heat exchange fluid in the feed stream in line 202 may be selected from one or more of toluene, methylcyclohexane n-pentane, and cyclopentane. In an aspect, both the organic heat exchange fluid in the feed stream in line 202 and the hydrocarbon feed stream in line 102 may comprise a similar hydrocarbon. In an exemplary embodiment, the organic heat exchange fluid in the feed stream in line 202 and the hydrocarbon feed stream in line 102 may comprise toluene. In another exemplary embodiment, the organic heat exchange fluid in the feed stream in line 202 and the hydrocarbon feed stream in line 102 may comprise a mixture of toluene and methylcyclohexane.

Referring back to the third effluent cooler 15, a thrice cooled third hydrogenated effluent stream is taken in line 145 from the third effluent cooler 15. The thrice cooled third hydrogenated effluent stream in line 145 may be further hydrogenated in the fourth hydrogenation reactor 150. In an embodiment, the thrice cooled third hydrogenated effluent stream in line 145 is combined with the fourth feed stream in line 106 to provide a combined fourth feed stream in line 146 which is charged to the fourth hydrogenation reactor 150 which may be a polishing reactor. In the fourth hydrogenation reactor 150, dehydrogenated hydrocarbon such as toluene present in the fourth feed stream in line 106 and in the thrice cooled third hydrogenated effluent stream in line 145 is hydrogenated in the presence of hydrogen and a hydrogenation catalyst to produce a fourth hydrogenated effluent stream comprising a hydrogenated hydrocarbon such as methylcyclohexane. The fourth hydrogenated effluent stream comprising the hydrogenated hydrocarbon such as methylcyclohexane is taken in line 152 from the fourth hydrogenation reactor 150. Hydrogen for the fourth hydrogenation reaction in the fourth hydrogenation reactor 150 may be present in the fourth hydrogenated effluent stream in line 142. Fresh make-up hydrogen may be supplemented to the fourth hydrogenation reactor 150, but this is not preferred.

Any suitable hydrogenation catalysts may be used in the fourth hydrogenation reactor 150. The fourth hydrogenation reactor 150 may comprise one or more of the hydrogenation catalyst as previously described. The fourth hydrogenation reactor 150 may comprise a similar or a different hydrogenation catalyst than the first hydrogenation reactor 120 and/or the second hydrogenation reactor 130 and/or the third hydrogenation reactor 140.

The fourth hydrogenation reactor 150 may be operated at a pressure of about 1034 kPa(g) (150 psig) to about 6895 kPa(g) (1000 psig), or about 2068 kPa(g) (300 psig) to about 3447 (500 psig). The fourth hydrogenation reactor 150 may be operated at an inlet temperature of about 204° C. (400° F.) to about 232° C. (450° F.). The fourth hydrogenation reactor 150 may be operated at an outlet temperature of about 232° C. (450° F.) to about 371° C. (700° F.). The fourth hydrogenation reactor 150 may be operated at similar or different operating pressure and temperature than the first hydrogenation reactor 120 and/or the second hydrogenation reactor 130 and/or the third hydrogenation reactor 140.

The fourth hydrogenation reactor 150 may be deemed a polishing reactor. The fourth hydrogenated effluent stream in line 152 exits the fourth reactor 150 at a temperature of about the outlet temperature of the fourth reactor 150. In an aspect, the fourth hydrogenated effluent stream in line 152 exits the fourth hydrogenation reactor 150 at the same temperature at which the third hydrogenated effluent stream in line 142 exits the third hydrogenation reactor 140. In another aspect, the fourth hydrogenated effluent stream in line 152 may exit the fourth hydrogenation reactor 150 at a lower temperature than the temperature at which the third hydrogenated effluent stream in line 142 exits the third hydrogenation reactor 140. In yet another aspect, the fourth hydrogenated effluent stream in line 152 may exit the fourth hydrogenation reactor 150 at a higher temperature than the temperature at which the third hydrogenated effluent stream in line 142 exits the third hydrogenation reactor 140. Heat can be recovered from the fourth hydrogenated effluent stream in line 152 which may be utilized in the process 101 or exported to other locations.

In an embodiment, the fourth hydrogenated effluent stream in line 152 is passed to the combined feed heat exchanger 110 to heat the combined first feed stream in line 108 by heat exchange. A cooled fourth hydrogenated effluent stream is discharged in line 154 from the combined feed heat exchanger 110 while the heated first combined feed stream is provided from the combined feed heat exchanger in line 112. The cooled fourth hydrogenated effluent stream in line 154 comprises hydrogenated hydrocarbon such as methylcyclohexane which is separated into a product stream in the purification section 151. In an embodiment, the purification section 151 comprises a high-pressure separator 160 and a stabilizer column 170.

In an embodiment, the cooled fourth hydrogenated effluent stream in line 154 is separated in the high-pressure separator 160 into a liquid stream comprising methylcyclohexane and a vapor stream comprising hydrogen. The cooled fourth hydrogenated effluent stream in line 154 may be further cooled in a cooler such an air cooler 155 and a twice cooled fourth hydrogenated effluent stream in line 156 is passed to the high-pressure separator 160. An overhead vapor stream comprising hydrogen is produced in line 162 from the high-pressure separator 160. The overhead vapor stream in line 162 may be recycled to the hydrogenation reactor section 111. The overhead vapor stream in line 162 may be compressed in a recycle compressor 163 to provide a compressed overhead stream in line 164 to provide hydrogen requirements.

From the bottom of the high-pressure separator 160, a hot liquid stream comprising a hydrogenated hydrocarbon such as methylcyclohexane is produced in line 165. In an aspect, the hot liquid stream in line 165 may be separated into a first hot liquid stream in line 166 and a second hot liquid stream in line 167. The second hot liquid stream in line 167 may be recycled to the hydrogenation reactor section 111. In an embodiment, the second hot liquid stream in line 167 may be pumped through a recycle pump 161 and the pumped second hot liquid stream in line 169 may be combined with the first feed stream in line 103 to provide the combined first feed stream in line 108. In an embodiment, the compressed overhead stream in line 164 may be mixed with a makeup hydrogen stream in line 174 to provide the hydrogen containing stream in line 179. In an aspect, the makeup hydrogen stream in line 174 may be the hydrogen feed stream for providing the hydrogen to the process 101. The hydrogen-containing stream in line 179 and the combined first feed stream in line 108 are heated in the combined feed heat exchanger 110 and charged to the first hydrogenation reactor 130 in the heated first feed line 112.

The first hot liquid stream in line 166 may be passed to the stabilizer column 170 to remove dissolved gases that may be present in the first hot liquid stream. The dissolved gases are separated in an overhead stream in line 171 from the stabilizer column 170. The stabilizer overhead stream in line 171 is cooled and passed to an overhead receiver 180. An off-gas stream comprising the dissolved gases may be taken in line 173 from the overhead receiver 180. A condensed liquid stream is taken in line 181 from the overhead receiver 180. A reflux stream is fed in line 182 and recycled back to the top of the stabilizer column 170. The entirety of the condensed liquid stream in line 181 may be refluxed to the stabilizer column 170 in the reflux line 182. Optionally, an overhead liquid stream may be taken in line 184 from the condensed liquid stream. In an embodiment, the overhead liquid stream in line 184 may comprise C₅-C₆ hydrocarbons.

A bottoms stream comprising hydrogenated hydrocarbon such as methylcyclohexane is produced in line 176 from the stabilizer column 170. A reboiling stream is taken in line 177 and reboiled in a reboiler 185 to provide a reboiled stream in line 177 which is recycled back to the bottoms of the stabilizer column 170. A product stream comprising hydrogenated hydrocarbon such as methylcyclohexane is taken in a product line 178 from the bottom of the stabilizer column 170. In an aspect, the product stream comprising hydrogenated hydrocarbon such as methylcyclohexane in line 178 may be stored and/or transported to a remote downstream or a second location for dehydrogenation to produce a hydrogen stream and a dehydrogenated stream comprising toluene. The hydrogen may be utilized at the remote downstream or second location and the dehydrogenated stream may be transported back to the process 101 and charged to the hydrogenation reactor section 111 in the feed line 102.

### Example

A simulation study was performed for the toluene-based ORC in the heat recovery unit 211. The toluene-based ORC was compared with a typical steam-based Rankin cycle in the heat recovery unit 211. In order to compare the performance to the steam-based Rankin cycle, air cooling was used with a 60° C. (140° F.) outlet temperature. The toluene liquid was pumped to the reactor effluent cooler, which provided over 80% of the reactor cooling as sensible heat. Toluene liquid from the reactor effluent cooler was discharged to the toluene vaporizer where the liquid toluene was pumped through the vaporizer on the reactor effluent, consuming about 10% of the reactor effluent heat and generating toluene vapor at a high pressure. It was found that toluene yielded up to 23% more power than steam. Although the mass flow of the toluene was 4.8 times the steam due to low heat of vaporization of toluene, the inlet volume of the toluene to the expander was 22% compared to the inlet volume to the expander for steam.

### Specific Embodiments

While the following is described in conjunction with specific embodiments, it will be understood that this description is intended to illustrate and not limit the scope of the preceding description and the appended claims.

A first embodiment of the present disclosure is a process of hydrogenating a hydrocarbons stream, comprising passing a dehydrogenated feed stream to a hydrogenation reactor; passing a hydrogen stream to the hydrogenation reactor; hydrogenating the dehydrogenated feed stream in the hydrogenation reactor in the presence of hydrogen and a hydrogenation catalyst to produce a hydrogenated effluent stream; and cooling the hydrogenated effluent stream by indirect heat exchange with an organic heat exchange fluid stream to provide a vaporized organic heat exchange fluid stream; and expanding the vaporized organic heat exchange fluid stream to provide electrical power. An embodiment of the present disclosure is one, any or all of prior embodiments in this paragraph up through the first embodiment in this paragraph, wherein the organic heat exchange fluid stream comprises toluene, methylcyclohexane or a mixture thereof. An embodiment of the present disclosure is one, any or all of prior embodiments in this paragraph up through the first embodiment in this paragraph further comprising passing the organic heat exchange fluid stream to an effluent cooler to heat exchange with the hydrogenated effluent stream to produce a heated organic heat exchange fluid stream; passing the heated organic heat exchange fluid stream to a vaporizer to heat exchange with the hydrogenated effluent stream to produce the vaporized organic heat exchange fluid stream; and passing the vaporized organic heat exchange fluid stream to an expander to generate the electrical power. An embodiment of the present disclosure is one, any or all of prior embodiments in this paragraph up through the first embodiment in this paragraph further comprising taking an expanded organic heat exchange fluid stream from the expander; cooling the expanded organic heat exchange fluid stream by heat exchange with the organic heat exchange fluid stream to produce a cooled organic heat exchange fluid stream; passing the cooled organic heat exchange fluid stream to a receiver to provide a condensed organic heat exchange fluid stream; and taking the organic heat exchange fluid stream from the condensed organic heat exchange fluid stream. An embodiment of the present disclosure is one, any or all of prior embodiments in this paragraph up through the first embodiment in this paragraph further comprising cooling a first hydrogenated effluent stream in a first effluent cooler by heat exchange with a first organic heat exchange fluid stream taken from the organic heat exchange fluid stream to produce a heated first organic heat exchange fluid stream; cooling a second hydrogenated effluent stream in a second effluent cooler by heat exchange with a second organic heat exchange fluid stream taken from the organic heat exchange fluid to produce a heated second organic heat exchange fluid stream; passing the heated first organic heat exchange fluid stream and the heated second organic heat exchange fluid stream to a heat exchange fluid separator to provide a vapor organic heat exchange fluid stream and a liquid organic heat exchange fluid stream; and passing the vapor organic heat exchange fluid stream to the expander. An embodiment of the present disclosure is one, any or all of prior embodiments in this paragraph up through the first embodiment in this paragraph further comprising cooling the first hydrogenated effluent stream in a first vaporizer by heat exchange with a first liquid organic heat exchange fluid stream taken from the liquid organic heat exchange fluid stream to produce a vaporized first organic heat exchange fluid stream; cooling the second hydrogenated effluent stream in a second vaporizer by heat exchange with a second liquid organic heat exchange fluid stream taken from the organic heat exchange fluid stream to produce a vaporized second organic heat exchange fluid stream; and passing the vaporized first organic heat exchange fluid stream and the vaporized second organic heat exchange fluid stream to the separator. An embodiment of the present disclosure is one, any or all of prior embodiments in this paragraph up through the first embodiment in this paragraph further comprising cooling a third hydrogenated effluent stream in a third effluent cooler by heat exchange with a third organic heat exchange fluid stream taken from the organic heat exchange fluid stream to produce a heated third organic heat exchange fluid stream; cooling the third hydrogenated effluent stream in a third vaporizer by heat exchange with a third liquid organic heat exchange fluid stream taken from the liquid organic heat exchange fluid stream to produce a vaporized third organic heat exchange fluid stream; and passing the heated third organic heat exchange fluid stream and the vaporized third organic heat exchange fluid stream to the separator. An embodiment of the present disclosure is one, any or all of prior embodiments in this paragraph up through the first embodiment in this paragraph further comprising superheating the vapor organic heat exchange fluid stream in a superheater by heat exchange with a third hydrogenated effluent stream to produce a superheated organic heat exchange fluid stream; and passing the superheated organic heat exchange fluid stream to the expander. An embodiment of the present disclosure is one, any or all of prior embodiments in this paragraph up through the first embodiment in this paragraph, wherein the third hydrogenated effluent stream is directly passed to the superheater to superheat the vapor organic heat exchange fluid stream. An embodiment of the present disclosure is one, any or all of prior embodiments in this paragraph up through the first embodiment in this paragraph further comprising passing the dehydrogenated feed stream to a first hydrogenation reactor to produce the first hydrogenated effluent stream; passing a cooled first hydrogenated effluent stream to a second hydrogenation reactor to produce a second hydrogenated effluent stream; and separating the second hydrogenated effluent stream to produce a product stream. An embodiment of the present disclosure is one, any or all of prior embodiments in this paragraph up through the first embodiment in this paragraph further comprising passing a cooled second hydrogenated effluent stream to a third hydrogenation reactor to produce the third hydrogenated effluent stream; passing a cooled third hydrogenated effluent stream to a fourth hydrogenation reactor to provide a fourth hydrogenated effluent stream; and separating the second hydrogenated effluent stream to produce the product stream An embodiment of the present disclosure is one, any or all of prior embodiments in this paragraph up through the first embodiment in this paragraph further comprising heat exchanging the second hydrogenated effluent stream with the dehydrogenated feed stream to heat the dehydrogenated feed stream and provide a cooled second hydrogenated effluent stream; separating the cooled second hydrogenated effluent stream to provide an overhead vapor stream and a bottoms liquid stream; taking a recycle liquid stream from the bottoms liquid stream; and recycling the recycle liquid stream and the overhead vapor stream to the first hydrogenation reactor with the dehydrogenated feed stream. An embodiment of the present disclosure is one, any or all of prior embodiments in this paragraph up through the first embodiment in this paragraph further comprising heat exchanging the recycle liquid stream and the overhead vapor stream with the second hydrogenated effluent stream before recycling. An embodiment of the present disclosure is one, any or all of prior embodiments in this paragraph up through the first embodiment in this paragraph further comprising compressing the overhead vapor stream to provide a compressed vapor stream; and heat exchanging the compressed vapor stream with the second hydrogenated effluent stream. An embodiment of the present disclosure is one, any or all of prior embodiments in this paragraph up through the first embodiment in this paragraph further comprising passing the bottoms liquid stream to a stabilizer column; and fractionating the bottoms liquid stream in the stabilizer column to produce a product stream comprising methylcyclohexane. An embodiment of the present disclosure is one, any or all of prior embodiments in this paragraph up through the first embodiment in this paragraph further comprising dehydrogenating the product stream at a downstream location to produce the dehydrogenated feed stream.

A second embodiment of the present disclosure is a process of hydrogenating a hydrocarbons stream, comprising passing a dehydrogenated feed stream to a hydrogenation reactor; passing a hydrogen stream to the hydrogenation reactor; hydrogenating the dehydrogenated feed stream in the hydrogenation reactor in the presence of hydrogen and a hydrogenation catalyst to produce a hydrogenated effluent stream; cooling the hydrogenated effluent stream by indirect heat exchange with an organic heat exchange fluid stream to provide a vaporized organic heat exchange fluid stream, wherein the organic heat exchange fluid stream comprises toluene, methylcyclohexane or a mixture thereof, and expanding the vaporized organic heat exchange fluid stream to provide electrical power. An embodiment of the present disclosure is one, any or all of prior embodiments in this paragraph up through the second embodiment in this paragraph further comprising passing the organic heat exchange fluid stream to an effluent cooler to heat exchange with the hydrogenated effluent stream to produce a heated organic heat exchange fluid stream; passing the heated organic heat exchange fluid stream to a vaporizer to heat exchange with the hydrogenated effluent stream to produce the vaporized organic heat exchange fluid stream; and passing the organic heat exchange fluid stream to an expander to generate the electrical power. An embodiment of the present disclosure is one, any or all of prior embodiments in this paragraph up through the second embodiment in this paragraph further comprising taking an expanded organic heat exchange fluid from the expander; cooling the expanded organic heat exchange fluid stream by heat exchange with the organic heat exchange fluid stream to produce a cooled organic heat exchange fluid stream; passing the cooled organic heat exchange fluid stream to a receiver to provide a condensed organic heat exchange fluid stream; and taking the organic heat exchange fluid stream from the condensed organic heat exchange fluid stream.

A third embodiment of the present disclosure is a process of hydrogenating a hydrocarbons stream, comprising passing a dehydrogenated feed stream to a hydrogenation reactor; passing a hydrogen stream to the hydrogenation reactor; hydrogenating the dehydrogenated feed stream in the hydrogenation reactor in the presence of hydrogen and a hydrogenation catalyst to produce a hydrogenated effluent stream; cooling the hydrogenated effluent stream by indirect heat exchange with a liquid organic heat exchange fluid stream to provide a vaporized organic heat exchange fluid stream; expanding the vaporized organic heat exchange fluid stream to provide electrical power; cooling an expanded organic heat exchange fluid stream to provide a condensed organic heat exchange fluid stream; and taking the liquid organic heat exchange fluid stream from the condensed organic heat exchange fluid stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of hydrogenating a hydrocarbons stream, comprising:
passing a dehydrogenated feed stream to a hydrogenation reactor;
passing a hydrogen stream to the hydrogenation reactor;
hydrogenating said dehydrogenated feed stream in the hydrogenation reactor in the presence of hydrogen and a hydrogenation catalyst to produce a hydrogenated effluent stream; and
cooling said hydrogenated effluent stream by indirect heat exchange with an organic heat exchange fluid stream to provide a vaporized organic heat exchange fluid stream; and
expanding said vaporized organic heat exchange fluid stream to provide electrical power.

2. The process of claim 1, wherein said organic heat exchange fluid stream comprises toluene, methylcyclohexane or a mixture thereof.

3. The process of claim 1 further comprising:
passing said organic heat exchange fluid stream to an effluent cooler to heat exchange with said hydrogenated effluent stream to produce a heated organic heat exchange fluid stream;
passing said heated organic heat exchange fluid stream to a vaporizer to heat exchange with said hydrogenated effluent stream to produce said vaporized organic heat exchange fluid stream; and
passing said vaporized organic heat exchange fluid stream to an expander to generate the electrical power.

4. The process of claim 3 further comprising:
taking an expanded organic heat exchange fluid stream from the expander;
cooling said expanded organic heat exchange fluid stream by heat exchange with said organic heat exchange fluid stream to produce a cooled organic heat exchange fluid stream;
passing said cooled organic heat exchange fluid stream to a receiver to provide a condensed organic heat exchange fluid stream; and
taking said organic heat exchange fluid stream from said condensed organic heat exchange fluid stream.

5. The process of claim 1 further comprising:
cooling a first hydrogenated effluent stream in a first effluent cooler by heat exchange with a first organic heat exchange fluid stream taken from said organic heat exchange fluid stream to produce a heated first organic heat exchange fluid stream;
cooling a second hydrogenated effluent stream in a second effluent cooler by heat exchange with a second organic heat exchange fluid stream taken from said organic heat exchange fluid to produce a heated second organic heat exchange fluid stream;
passing said heated first organic heat exchange fluid stream and said heated second organic heat exchange fluid stream to a heat exchange fluid separator to provide a vapor organic heat exchange fluid stream and a liquid organic heat exchange fluid stream; and
passing said vapor organic heat exchange fluid stream to an expander.

6. The process of claim 5 further comprising:
cooling said first hydrogenated effluent stream in a first vaporizer by heat exchange with a first liquid organic heat exchange fluid stream taken from said liquid organic heat exchange fluid stream to produce a vaporized first organic heat exchange fluid stream;
cooling said second hydrogenated effluent stream in a second vaporizer by heat exchange with a second liquid organic heat exchange fluid stream taken from said organic heat exchange fluid stream to produce a vaporized second organic heat exchange fluid stream; and
passing said vaporized first organic heat exchange fluid stream and said vaporized second organic heat exchange fluid stream to the separator.

7. The process of claim 6 further comprising:
cooling a third hydrogenated effluent stream in a third effluent cooler by heat exchange with a third organic heat exchange fluid stream taken from said organic heat exchange fluid stream to produce a heated third organic heat exchange fluid stream;
cooling said third hydrogenated effluent stream in a third vaporizer by heat exchange with a third liquid organic heat exchange fluid stream taken from said liquid organic heat exchange fluid stream to produce a vaporized third organic heat exchange fluid stream; and
passing said heated third organic heat exchange fluid stream and said vaporized third organic heat exchange fluid stream to the separator.

8. The process of claim 7 further comprising:
superheating said vapor organic heat exchange fluid stream in a superheater by heat exchange with a third hydrogenated effluent stream to produce a superheated organic heat exchange fluid stream; and
passing said superheated organic heat exchange fluid stream to the expander.

9. The process of claim 8, wherein said third hydrogenated effluent stream is directly passed to the superheater to superheat said vapor organic heat exchange fluid stream.

10. The process of claim 5 further comprising:
passing said dehydrogenated feed stream to a first hydrogenation reactor to produce said first hydrogenated effluent stream;
passing a cooled first hydrogenated effluent stream to a second hydrogenation reactor to produce a second hydrogenated effluent stream; and
separating said second hydrogenated effluent stream to produce a product stream.

11. The process of claim 10 further comprising:
passing a cooled second hydrogenated effluent stream to a third hydrogenation reactor to produce said third hydrogenated effluent stream;
passing a cooled third hydrogenated effluent stream to a fourth hydrogenation reactor to provide a fourth hydrogenated effluent stream; and
separating said fourth hydrogenated effluent stream to produce said product stream.

12. The process of claim 11 further comprising:
heat exchanging said fourth hydrogenated effluent stream with said dehydrogenated feed stream to heat said dehydrogenated feed stream and provide a cooled fourth hydrogenated effluent stream;
separating said cooled fourth hydrogenated effluent stream to provide an overhead vapor stream and a bottoms liquid stream;
taking a recycle liquid stream from said bottoms liquid stream; and
recycling said recycle liquid stream and said overhead vapor stream to the first hydrogenation reactor with said dehydrogenated feed stream.

13. The process of claim 12 further comprising:
heat exchanging said recycle liquid stream and said overhead vapor stream with said fourth hydrogenated effluent stream before recycling.

14. The process of claim 12 further comprising:
compressing said overhead vapor stream to provide a compressed vapor stream; and
heat exchanging said compressed vapor stream with said fourth hydrogenated effluent stream.

15. The process of claim 12 further comprising:
passing said bottoms liquid stream to a stabilizer column; and
fractionating said bottoms liquid stream in the stabilizer column to produce a product stream comprising methylcyclohexane.

16. The process of claim 12 further comprising:
dehydrogenating said product stream at a downstream location to produce said dehydrogenated feed stream.

17. A process of hydrogenating a hydrocarbons stream, comprising:
passing a dehydrogenated feed stream to a hydrogenation reactor;
passing a hydrogen stream to the hydrogenation reactor;
hydrogenating said dehydrogenated feed stream in the hydrogenation reactor in the presence of hydrogen and a hydrogenation catalyst to produce a hydrogenated effluent stream;
cooling said hydrogenated effluent stream by indirect heat exchange with an organic heat exchange fluid stream to provide a vaporized organic heat exchange fluid stream, wherein said organic heat exchange fluid stream comprises toluene, methylcyclohexane or a mixture thereof, and
expanding said vaporized organic heat exchange fluid stream to provide electrical power.

18. The process of claim 15 further comprising:
passing said organic heat exchange fluid stream to an effluent cooler to heat exchange with said hydrogenated effluent stream to produce a heated organic heat exchange fluid stream;
passing said heated organic heat exchange fluid stream to a vaporizer to heat exchange with said hydrogenated effluent stream to produce said vaporized organic heat exchange fluid stream; and
passing said organic heat exchange fluid stream to an expander to generate the electrical power.

19. The process of claim 16 further comprising:
taking an expanded organic heat exchange fluid from the expander;
cooling said expanded organic heat exchange fluid stream by heat exchange with said organic heat exchange fluid stream to produce a cooled organic heat exchange fluid stream;
passing said cooled organic heat exchange fluid stream to a receiver to provide a condensed organic heat exchange fluid stream; and
taking said organic heat exchange fluid stream from said condensed organic heat exchange fluid stream.

20. A process of hydrogenating a hydrocarbons stream, comprising:
passing a dehydrogenated feed stream to a hydrogenation reactor;
passing a hydrogen stream to the hydrogenation reactor;
hydrogenating said dehydrogenated feed stream in the hydrogenation reactor in the presence of hydrogen and a hydrogenation catalyst to produce a hydrogenated effluent stream;
cooling said hydrogenated effluent stream by indirect heat exchange with a liquid organic heat exchange fluid stream to provide a vaporized organic heat exchange fluid stream;
expanding said vaporized organic heat exchange fluid stream to provide electrical power;
cooling an expanded organic heat exchange fluid stream to provide a condensed organic heat exchange fluid stream; and
taking said liquid organic heat exchange fluid stream from said condensed organic heat exchange fluid stream.
